(19) United States
(12) Patent Application Publication
(10) Pub. No.: US 2009/0088868 A1
(43) Pub. Date: Apr. 2, 2009

(54) Low-profile lower leg prosthetic devices

(76) Inventor: Milo S. Collier, Longview, WA (US)

Correspondence Address: KOLISCH HARTWELL, P.C., 200 PACIFIC BUILDING, 520 SW YAMHILL STREET, PORTLAND, OR, 97204, US

(21) Appl. No.: 12/284,169

(22) Filed: Sep. 17, 2008

Related U.S. Application Data
(60) Provisional application No. 60/994,270, filed on Sep. 17, 2007.

(51) Int. Cl. A61F 2/64; A61F 2/66

(52) U.S. Cl. 623/47; 623/53; 623/55

(57) ABSTRACT

A lower leg prosthetic device includes an ankle assembly coupled to a foot assembly via a rigid pin extending through an oversized shaft having a pair of resilient bushings partially seated therein, which resiliently limit the relative pivot of the foot and ankle assemblies about axes orthogonal to that of the pin. A pair of cushions disposed between the assemblies forward and rearward of the pin resiliently limits relative pivot of the assemblies about the pin axis. The foot assembly includes an elongate, planar plantar member mounted to and extending forwardly of a support element having a comparatively lower elastic modulus. The foot assembly cooperates with the coupling assembly to absorb stride-related forces in a graduated manner by relative forward pivoting of the ankle assembly relative in a first phase of striding motion, and by upward flexion of the forward end of the plantar member in a second phase.

### CROSS-REFERENCE TO RELATED APPLICATION

This application claims priority from U.S. Provisional Patent Application Ser. No. 60/994,270, filed Sep. 17, 2007 entitled ANKLE MODULE FOR A PROSTHETIC DEVICE, the disclosure of which is incorporated herein by reference.

### TECHNICAL FIELD

The present disclosure is generally related to prosthetic devices, and more specifically to lower leg prosthetic devices.

### BACKGROUND

A prosthetic device, or prosthesis, is an artificial substitute for a part of the body, such as a limb. Numerous prostheses have been developed for this purpose, each trying to replicate the function and/or appearance of the missing body part. A lower limb prosthesis is the mechanical device by which an amputee's residual limb may interact with an activity surface. Lower leg prosthetic devices that replicate the function of a lower leg or foot, in particular, present several challenges, as such devices should be light enough to allow comfortable use and be aesthetically tolerable to a user while maintaining sufficient strength and flexibility to withstand stride-related forces encountered when walking, running, jumping, or participating in other athletic activities while wearing a prosthesis.

Further, prostheses for an individual with a Syme's amputation, or an amputation through the ankle joint, present additional challenges. For example, because the site at which a Syme's prosthesis is attached to a user—typically at or near the ankle portion of a natural foot—is so close to the ground, such prostheses adopt a low-profile configuration as compared with other lower leg prosthetic devices. However, traditional configurations of Syme's prostheses, such as those consisting mainly of a continuous plate that slopes generally downward from the attachment site to form heel and/or toe portions, either do not properly accommodate dorsi-flexion (upward flexion relative to a longitudinal axis corresponding to that of a natural foot), offer limited flexibility to pivot about axes orthogonal to a longitudinal axis, or otherwise do not accurately replicate the action of a natural ankle joint responsive to stride-related forces to the prosthesis.

Examples of lower leg prostheses may be found in U.S. Pat. No. 5,545,234 to Collier, Jr. and U.S. Pat. No. 5,800,570 to Collier, which are hereby incorporated by reference. These prostheses include an artificial shin structure for connection to an amputation socket or knee, or may further be coupled to an artificial knee structure, for use by above-the-knee amputees.

### SUMMARY

Lower limb prosthetic devices, such as a low-profile lower leg prosthetic device suitable for use by a user with a Syme's amputation, are described herein. The illustrated embodiment incorporates various components and systems configured to simulate the action of a natural ankle and a natural foot.

For example, the illustrated embodiment includes an ankle assembly adapted to be attached to a user, which is coupled to a foot assembly via a coupling arrangement that provides action similar to that of a natural ankle joint. In particular, a downwardly depending plate of the ankle assembly is captured between two spaced-apart, generally vertical arms of the foot assembly, and the coupling assembly includes a rigid pin mounted to and extending between the arms, through an oversized shaft bored through the plate. This configuration provides relative pivoting motion of the foot and ankle assemblies about the axis of the pin, and also provides relative pivoting motion of the assemblies about one or more axes generally orthogonal to the axis of the pin, such that the coupling assembly acts as a universal joint. The coupling assembly also includes a bushing arrangement, with two generally resilient, frustoconical bushings partially seated within the oversized shaft and interposed between the plate and the arms. The density and annular nature of the bushings resiliently limit relative pivoting of the foot and ankle assemblies about axes orthogonal to the pin axis, and a cushion arrangement with two resilient cushions disposed between the ankle and foot assemblies in areas generally forward and rearward of the pin, respectively, similarly limit relative pivoting of the assemblies about the pin axis.

The foot assembly of the illustrated embodiment also includes an elongate, generally planar plantar member extending along a longitudinal axis substantially corresponding to that of a natural foot, mounted to a comparatively shorter support element that extends forwardly from the rear end of the plantar member partially along the length of the plantar member. At about the region corresponding to the ball of a natural foot, the bottom surface of the support member, as it extends further forward, gradually inclines upward from the upper surface of the plantar member, such that a shelf member at the forward end of the support member section is spaced from the upper surface of the plantar member. The plantar and support members are rigid, but flexible, with the support member having a higher modulus of elasticity as compared with the plantar member. As such, the spacing of the shelf member and the different elastic moduli provide for a differentiated longitudinal dorsi-flexion of the foot assembly upon the application of a stride-related force; such that when the forward end of the plantar member flexes upward to a predetermined extent, the shelf member contacts the plantar member and absorbs additional torque, and resiliently limits further upward flexure of the plantar member. The stored energy is released at the end of a stride, propelling the user forward.

In the illustrated embodiment, the configuration of the foot assembly cooperates with the joint assembly to provide a low-profile lower leg prosthetic device that accommodates dorsi-flexion caused by stride-related forces, such as by graduated distribution of such forces first to the joint and then to the foot assembly, and also allows the device to simulate the flexibility of a natural ankle.

### BRIEF DESCRIPTION OF THE DRAWINGS

FIG. 1 is a side elevation view of an illustrative embodiment of a low-profile lower leg prosthetic device according to the present disclosure.

FIG. 2 is a fragmentary, partially sectioned view of the ankle module of FIG. 1, showing internal structure.

FIG. 3 is a sectional view of the ankle module taken along the lines 3-3 of FIG. 1.

### DETAILED DESCRIPTION

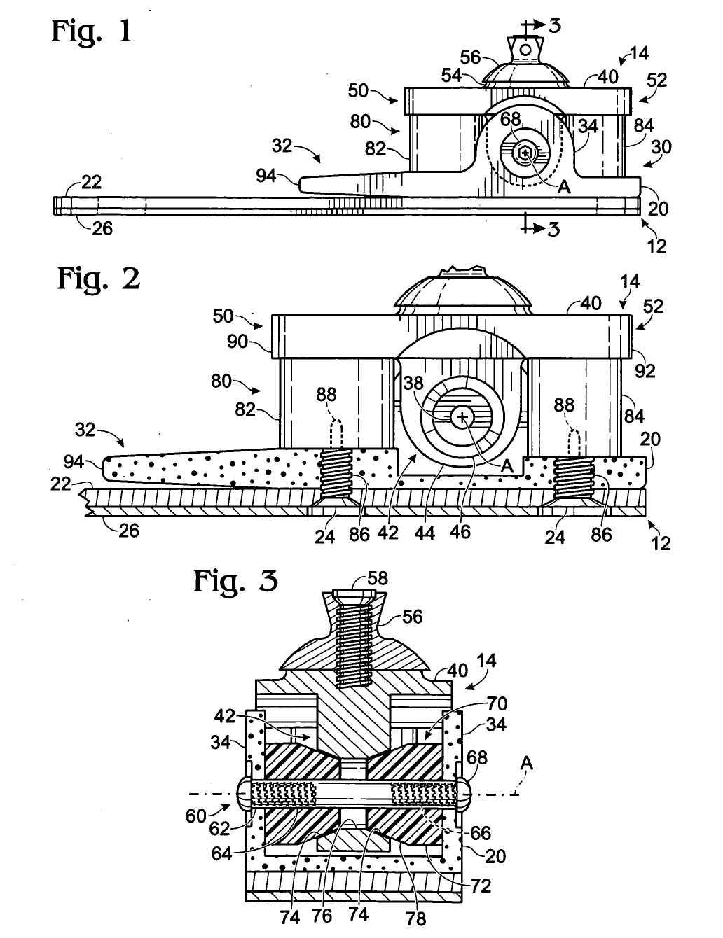

An illustrative embodiment of a low-profile lower leg prosthetic device is shown in FIGS. 1-3, with the device indicated generally at 10. As shown, the device includes a foot assembly 12 coupled with an ankle assembly 14. Although not shown, the prosthetic device will commonly be covered with a material such as rubber or polyurethane, or be fitted within a slipper fabricated from such a material, for example to simulate the appearance of a natural foot.

The foot assembly 12 of the device is shown to include a support element 20 and an elongate, generally flat (or planar) plate, or plantar member 22, with the plantar member extending forwardly from the support element along a longitudinal axis substantially corresponding to that of a natural foot. The plantar member is shown to be secured to the support element via threaded fasteners 24 (shown in FIG. 2). The foot assembly, and more particularly the plantar member, is adapted to longitudinally flex in response to application of a stride-related force to the foot assembly, as explained in further detail below. The plantar member is typically formed from a suitably rigid, yet resilient, material such as carbon graphite composite, and the support element is typically formed from a less resilient material such as glass-reinforced nylon, a nylon polymer, and so forth. An elongate cushion 26, typically fabricated from a porous, resilient material such as foam rubber, is applied to the plantar member, such as to further the spring action of the foot assembly, lessen heel impact, protect the bottom surface of the plantar member from damage or wear from directly engaging an activity surface, and so forth.

The support element 20 of the foot assembly includes a rearward heel section 30, a midfoot section 32 extending forwardly of the heel section along a length of the plantar member 22, and a pair of spaced arms 34 disposed generally between the heel and midfoot sections, which extend generally upward from the support element.

With reference also to FIG. 2, the ankle assembly 14 is shown to include a bearing element 40 that includes a coupling section 42 in the form of a downwardly depending plate 44, which has a shaft 46 bored therethrough. The bearing element 40, which is typically fabricated from a non-resilient material such as aluminum or some other metal, also includes forward and rear sections 50, 52, which extend forwardly and rearwardly, respectively, of the coupling section, and also includes a mount 54 for a coupling member 56 adapted to attach the device to a user. In the illustrated embodiment, coupling member 56 is shown in the form of a pyramid-type coupling, for example to be connected directly to a corresponding amputation socket of a user, such as a user with a Syme's amputation. However, other types of coupling members may instead be used, such as a coupling member with a lower or higher profile, or the device may be attached to other artificial leg structures such as a shank or shin structure, as appropriate for a user. Also, the pyramid coupling is shown to be secured to the bearing element via bolt 58, but alternative embodiments may include a coupling member that is unitary with, or integrated into, the bearing element or ankle assembly. Such variations are considered to be within the scope of this disclosure.

With reference also to FIG. 3, it can be seen that coupling section 42 of the ankle assembly is captured between arms 34 of the foot assembly, with the ankle assembly being movably coupled to the foot assembly to form an ankle joint via a coupling assembly that is indicated generally at 60. The coupling assembly includes a rigid pin 62 that is mounted to, and extends between, arms 34 of the support element, and through the oversized shaft 46 of the bearing element. The pin is typically formed of a material such as aluminum or some other metal. As such, the coupling assembly functions as an axle and permits the joint to pivot. More specifically, the coupling assembly permits pivoting motion of the ankle assembly relative to the foot assembly about the axis of the pin (indicated at A in FIGS. 1 and 3), at a location on the device roughly corresponding to that of the ankle of a natural foot.

As shown, pin 62 is shown to have a cylindrical exterior surface 64, with each end of the cylinder including a partially threaded interior surface 66 that is adapted to receive a socket head bolt 68 or similar fastener, which secure the ends of the pin through corresponding tight-fitting openings 38 in each of the arms. Although the illustrated pin configuration, with the cylinder extending through openings 38, may provide for ready disassembly of the device, such as for repairing, changing or replacing various components thereof, any suitable pin structure may be used that functions as an axle to permit such pivoting motion.

The coupling assembly is also shown in FIG. 3 to include a bushing arrangement 70 that consists of an opposing pair of resilient bushings 72. Each bushing is interposed between one of arms 34 and the coupling plate of the bearing element 40, and includes a central bore through which the pin extends. More particularly, each bushing is partially seated within the oversized shaft 46 of the coupling plate. As shown, the bushings are partially frustoconical, with the smaller end of each bushing seated within the shaft, which correspondingly includes two complementary, outwardly convex frustoconical surfaces 74 disposed on either side of a central, cylindrical surface 76. Optionally, fully frustoconical bushings may be used, or bushings that are otherwise continuously or partially tapered along the length of the central bore.

The diameter of the pin relative to that of the oversized shaft allows the joint to provide movement of the prosthetic device similar to the action provided by a natural ankle. For example, in addition to pivot about the axis of the pin, the coupling assembly also permits relative pivoting motion of the ankle and foot assemblies about one or more axes generally orthogonal to the axis of the pin, such as to simulate “twisting” of the ankle relative to the foot (pivoting about a generally vertical axis), “rocking” of the ankle from side to side relative to the foot (pivoting about a generally longitudinal axis), and so forth. In other words, the relative pivot of the ankle assembly is akin to rotary pivot of a universal joint, with the density of the resilient bushings dampening or otherwise limiting pivoting as stride-related forces are applied to the device. Flexion of the foot assembly relative to the ankle assembly may thus be accommodated by means of the annular nature of the bushings relative to the pin and oversized shaft.

As shown in FIGS. 1 and 2, the device further includes a cushion arrangement 80 that is adapted to dampen, or otherwise resiliently limit or restrict, relative pivoting of the ankle and foot assemblies about the axis of the pin (axis A). In particular, cushion arrangement 80 includes first and second resilient cushions 82, 84 interposed between the ankle assembly and the foot assembly in areas generally forward and rearward, respectively, of the pin 62 and arms 34. As shown, the region of the support element between the midfoot section 32 and the arms 34 is generally flat, with fasteners 24 each including a threaded portion 86 and a post portion 88 that projects generally upward from the surface of the support element, upon which the cushions are seated. Forward and rear sections 50, 52 of the bearing element include forward and rear plates 90, 92, which press downward against, and compress, the cushions, under the weight of a user.

The post portions prevent or otherwise restrain movement of the cushions, such as lateral movement, from their positions, and may thus have any suitable configuration. Optionally, although not shown in the illustrated embodiment, the post portions in some embodiments may extend further upward into the cushions, such as all the way to the top surface of the cushion, stopping at some point below the top surface, and so forth, such as to provide a mechanical limit to the extent of downward compression of the cushions, or otherwise to provide mechanical reinforcement to the cushions. For example, FIG. 3 shows that in the illustrated embodiment, the side portions of the bearing element are shaped to provide some vertical clearance relative to the top edges of the arms, such that the top edges represent a limit to the downward movement of the ankle assembly relative to the foot assembly. As such, the configuration of the post portions may provide a similar limit, or a different desired limit, of such movement. Similarly, the post portions may be of a desired diameter or cross-section. Some embodiments may alternatively or additionally include post portions disposed to abut the exterior surface of one or both cushions.

Because different individuals require different degrees of mobility and relative flexibility (i.e., an athlete requires more than a sedentary person), the materials that form the various resilient components may be chosen according to a user's particular needs. The cushions, as well as the bushings, may thus be fabricated of any suitably resilient material, such as polyurethane, rubber, a urethane/rubber composite, and so forth, as appropriate to provide a desired degree of resiliency. Further, each cushion or bushing may be of the same resiliency or of a different resiliency than the other in the pair, for example to provide a desired degree of resistive force against pivoting of the ankle assembly in a particular direction. This may be accomplished by choosing different materials, or, additionally in the case of the cushions, using cushions of different size or shape, and so forth. In the illustrated embodiment, the first, or forward, cushion 82 is less resilient than the second, or rear, cushion 84, for example to absorb the initial impact of heel-strike, but the variations discussed herein are considered to be within the scope of the disclosure. Also, the forward cushion 82 is of similar resiliency to each of the bushings 72 (which are the same resiliency as each other), but it is within the scope of this disclosure that the resiliencies of these components may be chosen as appropriate for a user.

As such, relative pivoting of the ankle and foot assemblies is permitted, and resiliently limited, via the joint configuration disclosed herein, wherein the rigid pin extends through the resilient bushings, which are in turn partially seated within the oversized shaft of the coupling plate of the bearing element. This configuration, by interposing resilient components between rigid components and/or by otherwise spacing rigid components from each other, avoids or reduces shear and other mechanical stress that otherwise would result from rigid components contacting each other as the device responds to stride-related forces.

Referring again to the foot assembly 12, the assembly is shown to be of composite construction, with the elongate cushion adhered, or otherwise applied or bonded, to the flat plantar member 22, which is in turn secured to the support element 20 via fasteners 24. Although the surface of the support element against which the flat plantar member is secured is generally complementarily flat, at about the region corresponding to the ball of a natural foot, the bottom surface of midfoot section 32, as it extends forwardly, gradually inclines upward from the plantar member, such that a shelf member 94 at the forward end of the midfoot section is spaced from the upper surface of the plantar member. As mentioned above, the support element is made of a material that is less resilient than the plantar member; or, in other words, the modulus of elasticity of the plantar member is greater than that of the support element. The different moduli of elasticity provide for a differentiated longitudinal flexion of the foot assembly upon the application of a stride-related force.

Typically, in a striding motion, a natural foot is subject to a considerable amount of force during dorsi-flexion, or when the foot bends upward relative to its length. If the stride of a foot is considered as starting at heel-strike, or the point at which the heel strikes the activity surface, to the point at which the toes leave the surface, the weight bearing area moves generally forward, from the heel to the toe section, as the foot moves through the stride. The flexion of a foot is generally greatest just before the foot is swung forward to take a step, when the individual's weight is borne by the toe region of the foot.

The illustrated device accommodates such forces during phases of a striding motion by allowing the device to flex while dispersing the force throughout (and storing the energy within) different sections of the device, generally in a graduated manner, as provided by the resilient nature of the joint of the foot and ankle assemblies and the differentiated longitudinal flexion of the foot assembly.

Upon heel-strike, the comparatively more resilient rear cushion absorbs the impact of the device upon the surface. When the individual continues moving forward, the plantar member first is generally horizontal relative to the surface, then begins to longitudinally flex upward at the forward section, as the heel section is lifted off the surface. The coupling assembly may accommodate forward pivoting of the ankle assembly relative to the foot assembly during a first phase of dorsi-flexion, with a second phase of dorsi-flexion accommodated by upward flexure of the forward end of the plantar member. During such a phase, as mentioned above, torque is loaded on the forward end of the plantar member, which corresponds to the toe section of a natural foot. The forward end thus flexes upwardly to accommodate the torque, with the spacing between the midfoot section and the plantar member allowing more of the length of the plantar member to accommodate the force, such that the device simulates the action provided by flexing of toes.

The degree and extent to which the bottom surface of the midfoot section inclines upward from the flat plantar member may be chosen such that the shelf member engages the plantar member upon a predetermined extent of flexure thereof; as such, the shelf member also may absorb stride-related forces as well as mechanically dampen, limit, or otherwise reduce, further upward flexure of the plantar member beyond the predetermined extent. Thus flexed, the foot assembly acts as a longitudinally differentiated energy storage spring that absorbs, and then releases, the forces encountered during striding motion, simulating a natural foot. Accordingly, at the end of a stride, the stored energy in the device is released, propelling the user forward.

Although the present invention has been shown and described with reference to the foregoing operational principles and preferred embodiments, it will be apparent to those skilled in the art that various changes in form and detail may be made without departing from the spirit and scope of the invention. For example, although the illustrated embodiment is in the form of a low-profile prosthesis suitable for use for an individual with a Syme's amputation, the components and concepts disclosed herein, such as the coupling assembly, the foot assembly, the ankle assembly, and so forth, may be embodied in other types of lower limb, and more specifically lower leg, prosthetic devices. The present invention is intended to embrace all such alternatives, modifications and variances that fall within the scope of the appended claims.

It is believed that the disclosure set forth above encompasses multiple distinct inventions with independent utility. While each of these has been disclosed in a particular form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

Inventions embodied in various combinations and subcombinations of features, functions, elements, and/or properties may be claimed through presentation of new claims in a related application. Such new claims, whether they are directed to a different invention or directed to the same invention, whether different, broader, narrower or equal in scope to the original claims, are also regarded as included within the subject matter of the inventions of the present disclosure.

What is claimed is:

1. A prosthetic device comprising:
a foot assembly that includes a rearward heel section and an elongate plantar member extending forwardly therefrom along a longitudinal axis substantially corresponding to a natural foot, the heel portion further including a pair of spaced arms;
an ankle assembly that includes a coupling member adapted to attach the prosthetic device to a user, the ankle assembly further including a coupling section having an oversized shaft therethrough and extending between the spaced arms, the ankle assembly being coupled to the foot assembly via a coupling assembly that includes a pin mounted to the arms and extending through the shaft, the surface of the pin being spaced from the surface of the shaft to permit relative pivoting of the ankle and foot assemblies about an axis substantially orthogonal to the axis of the pin;
a bushing arrangement having a pair of resilient bushings, with each bushing being at least partially seated in the shaft such that each bushing is interposed between one of the pair of spaced arms and the coupling section, whereby the bushing arrangement is adapted to resiliently limit said pivoting motion of the ankle assembly.

2. The prosthetic device of claim 1, wherein each bushing includes a central bore through which the pin extends.

3. The prosthetic device of claim 2, wherein the central bore extends from a first end of each bushing to a second end that is smaller than the first end, and wherein the second end of each bushing is seated in the shaft.

4. The prosthetic device of claim 3, wherein each bushing is substantially frustoconical.

5. The prosthetic device of claim 2, wherein the surface of the shaft includes opposing complementarily shaped regions each adapted to receive at least a portion of a bushing.

6. The prosthetic device of claim 1, wherein the coupling assembly also permits pivoting motion of the ankle assembly relative to the foot assembly about the axis of the pin.

7. The prosthetic device of claim 6, further including a cushion arrangement having first and second resilient cushions interposed between the ankle assembly and the foot assembly in areas generally forward and rearward of the pin, respectively, which are adapted to resiliently limit the pivoting motion of the ankle assembly about the axis of the pin.

8. The prosthetic device of claim 7, wherein the first and second cushion are differently resilient.

9. The prosthetic device of claim 8, wherein the second cushion is more resilient than the first cushion.

10. The prosthetic device of claim 7, wherein the cushions are further adapted also to resiliently limit the limit relative pivoting of the ankle and foot assemblies about an axis substantially parallel to the longitudinal axis of the plantar member.

11. The prosthetic device of claim 1, wherein the plantar member has a first predetermined modulus of elasticity and is adapted to longitudinally flex in response to application of a stride-related force to the foot assembly, and wherein the foot assembly further includes a shelf member disposed forward of the heel portion and projecting above a portion of the plantar member, the shelf member having a second predetermined modulus of elasticity less than the first predetermined modulus of elasticity and being adapted to limit the longitudinal flex of the plantar member.

12. The prosthetic device of claim 11, wherein the plantar member is substantially planar.

13. A prosthetic device comprising:
an ankle assembly that includes a coupling member adapted to attach the prosthetic device to a user;
a foot assembly coupled to the ankle assembly and including:
an elongate, substantially planar plantar member having a first predetermined modulus of elasticity and adapted to longitudinally flex in response to application of a stride-related force to the foot assembly; and
a support element having a second predetermined modulus of elasticity less than the first predetermined modulus of elasticity, and further including a shelf member spaced from the plantar member for selective engagement therewith upon a predetermined extent of flexure of the plantar member.

14. The prosthetic device of claim 13, wherein the shelf member is spaced above a portion of the plantar member.

15. The prosthetic device of claim 14, wherein the shelf member is adapted to limit the upward longitudinal flex of the plantar member by engaging the portion of the plantar member above which the shelf member is spaced upon a predetermined extent of upward flexure of the plantar member.

16. The prosthetic device of claim 13, wherein the foot assembly is coupled to the ankle assembly via a coupling assembly that includes a pin that extends through a shaft of the ankle assembly, thereby permitting relative pivoting motion of the ankle and foot assemblies about the axis of the pin.

17. The prosthetic device of claim 16, wherein the surface of the pin is spaced from the surface of the shaft, thereby permitting relative pivoting motion of the ankle and foot assemblies about an axis substantially orthogonal to the axis of the pin.

18. The prosthetic device of claim 17, further including a bushing arrangement having a resilient bushing mounted between each arm and the coupling section adapted to resiliently limit said pivoting motion of the ankle assembly.

19. The prosthetic device of claim 17, further including a cushion arrangement interposed between the ankle and foot assemblies that is adapted to resiliently limit the pivoting motion of the ankle assembly about the axis of the pin.

20. The prosthetic device of claim 13, further including a cushion arrangement having first and second resilient cushions interposed between the ankle assembly and the foot assembly in areas generally forward and rearward of the pin, respectively, that is adapted to resiliently limit the pivoting motion of the ankle assembly about the axis of the pin.

21. The prosthetic device of claim 20, wherein the foot assembly further includes one or more post portions projecting upwardly from the support member and disposed to restrain lateral movement of the cushions.

22. The prosthetic device of claim 21, wherein the plantar member is secured to the support element via a pair of fasteners, of which the post portions form the upper portions.

23. The prosthetic device of claim 21, wherein the cushions are seated on the post portions.

24. A low-profile lower leg prosthetic device, comprising:
a foot assembly including an elongate, substantially planar plantar member extending along a longitudinal axis substantially corresponding to a natural foot and having a first predetermined modulus of elasticity, and a support element mounted to and extending partially along the length of the plantar member, the support element having a second predetermined modulus of elasticity less than the first predetermined modulus of elasticity,
an ankle assembly coupled to the foot assembly via a via a coupling assembly that permits relative pivoting of the ankle and foot assemblies about an axis corresponding to that of the ankle of a natural foot, the ankle assembly further being configured to attach the device to a user;
a cushion arrangement interposed between the ankle and foot assemblies and adapted to resiliently limit the relative pivoting motion thereof;
wherein the foot assembly cooperates with the coupling assembly to absorb, store, and release stride-related forces applied to the device in a graduated manner by forward pivoting of the ankle assembly relative to the foot assembly about the axis of a pin in a first phase of striding motion, and by upward flexion of the forward end of the plantar member in a second phase of striding motion.
